(19) United States
(12) Patent Application Publication
(10) Pub. No.: US 2026/0327607 A1
(43) Pub. Date: Oct. 22, 2026

(54) REVERSE TRANSCRIPTION DURING TEMPLATE EMULSIFICATION

(71) Applicant: ILLUMINA, INC., San Diego, CA (US)

(72) Inventors: Kristina Fontanez, Arlington, MA (US); Robert Meltzer, Framingham, MA (US); Yi Xue, Shrewsbury, MA (US); Christopher D'amato, Wellesley, MA (US); Sepehr Kiani, Watertown, MA (US)

(73) Assignee: ILLUMINA, INC., San Diego, CA (US)

(21) Appl. No.: 19/570,381

(22) Filed: Mar. 18, 2026

Related U.S. Application Data
(63) Continuation of application No. 17/740,998, filed on May 10, 2022, now abandoned.
Application No. 17/740,998 is a continuation of application No. 17/146,974, filed on Jan. 12, 2021, now abandoned.
(60) Provisional application No. 62/960,283, filed on Jan. 13, 2020.

(51) Int. Cl. C12N 15/10

(52) U.S. Cl. CPC C12N 15/1075; C12N 15/1096; C12N 15/1003

(57) ABSTRACT

Methods to emulsify cells and/or mRNA with reverse transcriptase at a temperature such that the reverse transcriptase begins making cDNA during the emulsification

This is a continuation application of U.S. patent application Ser. No. 17/740,998, filed May 10, 2022, which is a continuation application of U.S. patent application Ser. No. 17/146,974, filed Jan. 12, 2021, now abandoned, which claims priority to U.S. Provisional Patent Application No. 62/960,283, filed Jan. 13, 2020, the disclosures of which are incorporated herein by reference.

### TECHNICAL FIELD

The disclosure relates to tools for understanding gene expression and biology.

### BACKGROUND

In living organisms, genetic information is stored in DNA. Genes in the DNA are transcribed into messenger RNA (mRNA), which is translated into proteins. Proteins play critical functional and structural roles in living organisms. For example, most enzymes are made of proteins, and those enzymes catalyze the metabolic reactions that keep us alive. It is also enzymes that copy DNA into mRNA. Proteins are also structural, and constitute the essential fibers of muscles, the predominant material of hair, as well as basic structural linkages within the cytoskeleton. Essentially, all such proteins are made by translating an mRNA into the protein. In fact, one mRNA can serve as the template for synthesizing multiple copies of a protein.

Because living cells change in response to different environmental conditions, nutrient availability, and even intra-cellular signaling, the cells need different proteins at different times. It is beneficial to the cell's ability to change that any given mRNA is short-lived. It is thought that most mRNA molecules have a lifetime measured in seconds or minutes. The essential and ephemeral nature of mRNA presents a challenge to biological understanding. On the one hand, the mRNAs that are present in a cell at a given moment could reveal much about how the cell is responding to a pathogen, or a drug, or to age-specific developmental changes. On the other hand, in any attempt to remove a cell from its natural environment and study the mRNAs present, time is of the essence. Those mRNAs begin to degrade within seconds or minutes. As time is spent in the laboratory to set up a clinical or research assay, the very molecular ingredients of the cell to be studied begin to decay and the information they represent is lost.

### SUMMARY

The disclosure provides methods for reverse transcribing mRNA into complementary DNA (cDNA) while simultaneously isolating cells into aqueous partitions. Methods of the disclosure provide for the very rapid capture of the information in mRNA into cDNA, which is more stable than mRNA. The cDNAs are made immediately as the sample is emulsified into droplets. Methods of the disclosure make use of particles that serve as templates for making a large number of monodisperse emulsion droplets simultaneously in a single tube or vessel. By adding cells into an aqueous mixture that includes a plurality of hydrogel template particles, layering oil over the aqueous phase, and vortexing or pipetting the tube, the particles serve as templates while the shear force of the vortexing or pipetting causes the formation of water-in-oil monodisperse droplets with on particle in each droplet. Reverse transcription reagents can be included in the initial mixture, allowing reverse transcriptase to begin simultaneously with shearing the water/oil mixture to form the emulsions. Making cDNAs from the RNAs immediately during the first stage of the droplet-making process preserves the information present as mRNA in the original cells. The disclosure provides suitable reagents and conditions for successfully reverse transcribing mRNA into cDNA while isolating a plurality of cells into monodisperse droplets in a single tube.

Because the cDNA is made simultaneously with mixing the emulsions in the tube, important biological information is not lost due to the short lifetime of RNA molecules in living cells. Because the information of mRNA is preserved as cDNA, methods of the disclosure provide an additional useful tool for understanding the phenotype and gene expression of a given cell at any time. In fact, the cDNA can be amplified by, e.g., polymerase chain reaction, into a plurality of stable DNA amplicons that can be stored or studied under a variety of conditions or methods. Methods of the disclosure are well-suited to making DNA libraries suitable for sequencing on a next-generation sequencing (NGS) instrument.

An insight of the disclosure is that a plurality of droplets can be made in a single tube at a temperature and/or at a mixing speed that promote cDNA synthesis. For example, by mixing at about 50 degrees C and/or at about 500 rpm, methods can successfully initiate cDNA synthesis while, in the single tube, forming the droplets that contain the cells thereby isolated into individual aqueous partitions. Thus, methods of the disclosure provide important tools for basic biology, clinical research, and patient testing.

In certain aspects, the disclosure provides a library preparation method. The method includes preparing a mixture that includes cells and reagents for reverse transcription and vortexing or optionally pipetting the mixture. During the vortexing (or pipetting), the mixture partitions into aqueous droplets that each essentially include zero or one cell, the cells are lysed to release mRNA into the droplets, and reverse transcriptase copies the mRNA into cDNAs. The method preferably further includes amplifying the cDNAs into a library of amplicons. Preferably the mixture includes particles such that, during vortexing, the particles template the formation of the droplets. The particles may be gels that include the reagents therein. The mixture may be aqueous and the method may include adding an oil onto the mixture prior to the vortexing/pipetting. The method may include, during the vortexing, heating the mixture to a temperature that promotes activity of the reverse transcriptase (e.g., between about forty and about fifty degrees C). The mixture is preferably sheared by any suitable mechanism or device, such as a benchtop vortexer or shaker, a pipette (e.g., micropipette), a magnetic or other stirrer or similar.

In certain embodiments, the particles are linked to capture oligos that have a free, 3′ poly-T region. The particles may also include cDNA capture oligos that have 3′ portions that hybridize to cDNA copies of the mRNA. The 3′ portions of the cDNA capture oligos may include gene-specific sequences or oligomers. The oligomers may be random or “not-so-random” (NSR) oligomers (NSROs), such as random hexamers or NSR hexamers. The particles may be linked to capture oligos that include one or more handles such as primer binding sequences cognate to PCR primers that are used in the amplifying step or the sequences of NGS sequencing adaptors. The cDNA capture oligos may include template switching oligos (TSOs), which may include poly-G sequences that hybridize to and capture poly-C segments added during reverse transcription.

In some embodiments, the vortexing is performed on a vortexing instrument, e.g., which vortexes the mixture at a suitable rate such as between about two hundred and about seven hundred rpm (preferably about 500 rpm). The vortexing instrument may include a heater that heats the mixture during vortexing.

The mixture may be pre-prepared with a plurality of template particles at a number to capture a suitable target number of cells. For example, the mixture may initially include thousands, tens of thousands, hundreds of thousands, millions, or at least about 10 million template particles. Methods may be used to capture and partition any number of cells such as thousands, tens of thousands, hundreds of thousands, millions, or at least about 10 million cells. Each of the particles may contain some of the reagents for reverse transcription. The particles may be used to template the formation of monodisperse droplets. Preferably, each of the particles serves as a template to initiate formation of aqueous monodisperse droplets in oil, in which each droplet comprises one particle. The particles may be hydrogel particles and may include, for example, polyacrylamide (PAA) or polyethylene glycol (PEG).

Aspects of the disclosure provide a sample preparation method. The method includes preparing, in a sample vessel, an aqueous mixture that includes nucleic acids and polymerase enzymes. An oil is added to the sample vessel, and the method includes shaking or vortexing the sample vessel to simultaneously: (i) partition the aqueous mixture into droplets surrounded by the oil and (ii) synthesize a DNA copy of at least one of the nucleic acids with the polymerase during the shaking. The nucleic acids may initially be in cells and the shaking step may cause droplets to form that contain the cells. The method may include lysing the cells within the droplets to release the nucleic acids into the droplets. Lysing may be done by adding a lytic agent to the vessel (such as a detergent like sodium dodecyl sulfate (SDS)). In some embodiments, the vessel is heated to a temperature that promotes reverse transcription. It may be found that detergent, heat, and shaking work in combination to lyse the cells. In preferred embodiments, the nucleic acids include mRNA and the polymerase enzymes include reverse transcriptase enzymes.

Preferably the aqueous mixture includes a plurality of template particles, and shaking the sample vessel causes each template particle to serve as a template in the formation of one of the droplets. The nucleic acids may initially be in cells and the shaking step may cause droplets to form such that each of the droplet contains one template particle and one or zero cells. The method may include lysing the cells within the droplets to release the nucleic acids into the droplets and the method may include, during the shaking step, heating the aqueous mixture to a temperature that promotes reverse transcription.

In certain embodiments, the template particles are linked to capture oligos, which are linked to the template particles at their 5′ ends, and in which 3′ ends of the capture oligos include a poly-T sequence. Each of the template particles may contain some of the reverse transcriptase enzymes. The method may include, after the adding step, loading the sample vessel into an instrument that performs the shaking step. In some embodiments, during the shaking: the droplets form, cells are lysed within the droplets to release the nucleic acids, template particles capture the nucleic acids, and the polymerase enzymes synthesize the DNA copies.

The aqueous mixture may include a plurality of template particles (e.g., hydrogel particles), and the method may include, after the adding step, loading the sample vessel into an instrument that performs the shaking step and wherein shaking the sample vessel causes each template particle to serve as a template in the formation of one of the droplets. The nucleic acids may initially be in cells and the shaking step may cause droplets to form in which each of the droplets contains one template particle and one or zero cells. In some embodiments: the nucleic acids are mRNAs in cells in the aqueous mixture; the droplets contain the cells; the polymerase enzymes are provided in template particles within the aqueous mixture; and the template particles serve as template to cause formation of the droplets during the shaking. The method may include-after partitioning the aqueous mixture into the droplets-lysing the cells to release the mRNAs into the droplets. In certain embodiments the template particles are bound to capture oligos that capture the mRNAs and prime extension reactions by which the polymerase enzymes copy the mRNAs.

### BRIEF DESCRIPTION OF THE DRAWINGS

FIG. 1 diagrams a library preparation method.

FIG. 2 shows a mixture that includes cells and reagents for reverse transcription.

FIG. 3 shows loading an 8-tube strip into an instrument for vortexing.

FIG. 4 shows the droplets formed during vortexing.

FIG. 5 is a detail view of a droplet according to certain embodiments.

FIG. 6 is a photomicrograph showing a plurality of PAA particles.

FIG. 7 shows an embodiment in which the particles are linked to capture oligos.

FIG. 8 shows a cDNA linked to a particle.

FIG. 9 shows a first sense copy of the cDNA.

FIG. 10 shows the antisense copy that is made by extending the free forward primer.

FIG. 11 shows the sense copy of the original mRNA.

FIG. 12 diagrams a sample preparation method.

FIG. 13 shows results from performing methods of the disclosure.

### DETAILED DESCRIPTION

The disclosure generally relates to single-tube “direct to sequencing library” methods that can be used to isolate cells into fluid partitions (e.g., droplets) while also reverse transcribing RNA into cDNA while isolating the cells into the partitions. In some embodiments, premade particles, such as hydrogel particles, serve as templates that cause water-in-oil emulsion droplets to form when mixed in water with oil and vortexed or sheared. For example, an aqueous mixture can be prepared in a reaction tube that includes template particles and target cells in aqueous media (e.g., water, saline, buffer, nutrient broth, etc.). An oil is added to the tube, and the tube is agitated (e.g., on a vortexer aka vortex mixer). The particles act as template in the formation of monodisperse droplets that each contain one particle in an aqueous droplet, surrounded by the oil.

The droplets all form at moment of vortexing-essentially instantly as compared to the formation of droplets by flowing two fluids through a junction on a microfluidic chip. Each droplet thus provides an aqueous partition, surrounded by oil. An important insight of the disclosure is that the particles can be provided with reagents that promote useful biological reactions in the partitions and even that reverse transcription can be initiated during the mixing process that causes the formation of the partitions around the template droplets. Moreover, the pre-templated instant partitions may be formed while the reaction mixture is being heated to a temperature that promotes activity of reverse transcriptase. In fact, data show mixing conditions and particle compositions that promote successful copying of mRNA into cDNA during mixing of the mixture to form the pre-templated instant partitions.

Methods of the disclosure are useful in making a cDNA library. A cDNA library may be a useful way to capture and preserve information from RNAs present in a sample. For example, a sample that includes one or more intact cells may be mixed with template particles to form a partition (e.g., droplet) that includes the cell. The cell can be lysed and mRNAs can be reverse transcribed into cDNAs in the droplet during the mixing stage that forms the partitions. Similarly, a sample that includes cell-free RNA can be mixed with oligo-linked template particles and mixed (e.g., shaken, vortexed, or sheared) to form droplets while simultaneously beginning the transcribe the RNA to cDNA. Whether starting with whole cells or cell-free RNA, the result is the formation of droplets that include cDNA copies of the starting RNA. Because the cDNA is more stable than RNA (e.g., cDNA does not include 2′ hydroxyl groups that autocatalyze the molecule's own hydrolysis), the droplets provide a stable cDNA library that may be used in downstream assays to study the RNA content of the starting sample.

Forming the cDNAs while initially forming the droplets avoids problems caused by the ephemeral nature of mRNA. Sample preparation and library preparation methods of the disclosure improve the ability of laboratory techniques to study RNA compositions of a sample. In fact, cells can be sequestered into aqueous partitions while also, simultaneously copying the mRNAs into stable cDNA that may be stored and studied downstream.

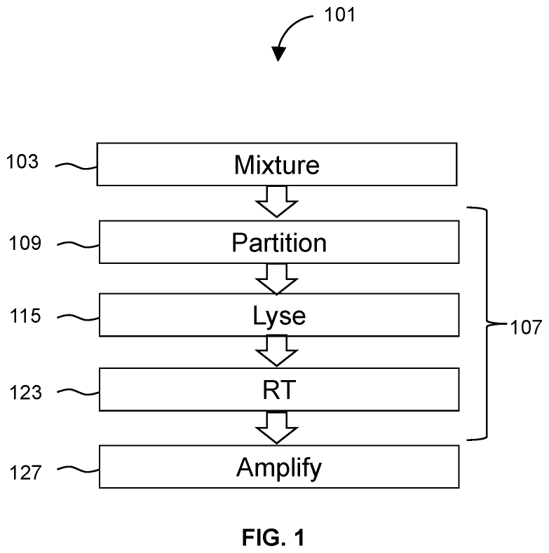

FIG. 1 diagrams a library preparation method 101. The method includes preparing 103 a mixture that includes cells and reagents for reverse transcription. While any suitable order may be used, it may be useful to provide a tube that includes template particles. The template particles may be provided in an aqueous media (e.g., saline, nutrient broth, water) or dried to be rehydrated at time of use. A sample may be added into the tube—e.g., directly upon sample collection from a patient, or after some minimal sample prep step such as spinning whole blood down, re-suspending peripheral blood monocytes (PBMCs), and transferring the PBMCs into the tube. Preferably an oil is added to the tube (which will typically initially overlay the aqueous mixture). The method 101 then includes vortexing 107 or pipetting the mixture to shear the fluid causing partitioning. It may be found that during the vortexing: the mixture partitions into the aqueous droplets within about 5 to about 50 seconds, and then the cells are lysed within about 30 seconds to about a few minutes, and then the reverse transcriptase begins to copy the mRNA.

During the vortexing, several things are accomplished. The mixture partitions 109 into aqueous droplets that each include zero or one cell. When the sample includes whole cells such as PBMCs, the cells are lysed 115 to release mRNA into the droplets. The lysing 115 is an optional step, as the method 101 may be used where the original sample includes cell-free RNA. Additionally, reverse transcriptase copies 123 the mRNA into cDNAs. Lysis may be performed chemically (e.g., using micelles to deliver lysis agents), by activated chemistry (e.g., thermal, light, etc), and/or enzymatically (heat activated). A mix of micelle/chemical plus heat-activated enzymes has been tested.

Embodiments of the disclosure employ chemical lysis methods including, for example, micelle-based methods. Methods may include using micelles to deliver suitable lysis agents.

Suitable lysis agents include Sarkosyl, SDS, Triton X-100. One or more surfactants is used to micellize the lysis agent into the oil phase. Suitable surfactants for creating micelles may include, for example Ran or ionic Krytox. It may be useful to use a super-concentrated co-solvent to aid dissolution of the lysis agent. Some embodiments use a combination of fluoro-phase surfactant Krytox 157-FSH (acidic form) or neutralized form (ammonium counter-ion, potassium counterion or sodium counter-ion) in 0.05%-5% in Novec 7500 or 7300 or 7100 or Fuorinert to form micelles that include a sysis agent such as Sarkosyl or SDS at 0.05%-5%. In certain emobdiments, a fluoro-phase surfactant such as Perfluorpolyether PEG-conjugates is used with a non-ionic Isysis agent such as Triton-X 100 or IGEPAL at 0.05%-2%. Fluorocarbon based oil system may be used, e.g., 3M Novec HFE (e.g. HFE 7000, 7100, 7200, 7300, 7500, 7800, 8200) or 3M Fluorinert (e.g. FC-40, −43, −70, −72, −770−3283−0.3284). Embodiments may use surfactant for fluorocarbon based oil, e.g., commercially available compounds such as Chemour Krytox 157FSH, Chemour Capstone etc. Ionic type fluorophase surfactants may include Perfluoroalkyl carboxylates, Perfluoroalkyl sulfonates, Perfluoroalkyl sulfates, Perfluoroalkyl phosphates, Perfluoropolyether carboxylates, Perfluoropolyether sulfonates, or Perfluoropolyether phosphates. Non-ionic type fluorophase surfactant may include Perfluoropolyether ethoxylates or Perfluoroalkyl ethoxylates. A silicone based oil system may be used such as polydimethylsiloxane (PDMS) with viscosity range between 0.5-1000 cst. Suitable surfactant for silicone based oil may be used such as Gelest Reactive Silicones, Evonik ABIL surfactant, etc. An ionic type silicone phase surfactant may be carboxylate terminated PDMS or Amine terminated PDMS. A non-ionic type silicone phase surfactant may be hydroxyl terminated PDMS or PEG/PPG functionalized PDMS. A hydrocarbon based oil system may use heavy alkane hydrocarbons with carbon atoms number greater than 9. The oil could include a single compound or a mixture from multiple compounds. For example, tetradecane, hexadecane, mineral oil with viscosity range between 3 to 1000 cst. Suitable surfactant for hydrocarbon based oil (ionic) may include Alkyl carboxylates, Alkyl sulfates, Alkyl sulfonates, Alkyl phosphates or (non-ionic) PEG-PPG copolymers (e.g. Pluronic F68, Pluronic F 127, Pluronic L121, Pluronic P123), PEG-alkyl ethers (e.g. Brij L4, Brij 58, Brij C10), PEG/PPG functionalized PDMS (e.g. Evonik ABIL EM90, EM180), Sorbitan derivatives (e.g. Span-60, Span-80, etc.), or Polysorbate derivatives (e.g. Tween-20, Tween 60, Tween 80). To achieve best micellization/co-dissolution performance and minimum disruption of water-in-oil droplet interface, the general rule of thumb for lysis agent/oil phase surfactant combination is as follow: (i) an ionic type lysis agent is preferred for combination with ionic oil phase surfactant, such lysis agent may include but not limited to: SDS, Sarkosyl, sodium deoxycholate, Capstone FS-61, CTAB; (ii) a non-ionic type lysis agent is preferred for combination with non-ionic oil phase surfactant, such lysis agent may include but not limited to: Triton X-100, Triton X-114, NP-40, Tween-80, Brij 35, Octyl glucoside, octyl thioglucoside; and/or (iii) a zwitterionic type lysis agent may be used in combination with either ionic or non-ionic oil phase surfactant, such lysis agent may include but not limited to: CHAPS, CHAPSO, ASB-14, ASB-16, SB-3-10, SB-3-12.

As shown, two important phenomena are accomplished during the vortexing 107 step: aqueous partitions form 109 and reverse transcription 123 occurs.

Importantly, a plurality (e.g., thousands, tens of thousands, hundreds of thousands, millions, or tens of millions or more) of aqueous partitions are formed 109 essentially simultaneously. Results have shown that this consistently works. It may be preferable to use template particles (e.g., a corresponding number of hydrogel particles that serve as templates to the formation of droplets). Reagents may be provided to promote cell lysis or initiate reverse transcription. Once the vortexing 107 step has been performed, at least one of the droplets will have at least one cDNA copy of an RNA from the starting sample. For background overview, see generally Gubler, 1983, A simple and very efficient method for generating cDNA libraries, Gene 25(2-3): 263-9 and Figueiredo, 2007, Cost effective method for construction of high quality cDNA libraries, Biomolecular Eng 24:419-421, both incorporated by reference. Preferably, one or a plurality of the droplets will each have a plurality of cDNAs that include droplet-specific oligonucleotide barcodes for a plurality of corresponding RNAs that were partitioned into the droplets by the partitioning 109. Forming the cDNA(s) may include attaching amplification primer-binding sites (such as first and second universal priming sequences at the ends of the cDNAs), and the method 101 optionally includes amplifying 127 the cDNA(s) into amplicons, which may be stored or analyzed. For example, the amplicons may be sequenced using a sequencer such as a next-generation sequencing (NGS) instrument.

To prepare 103 the mixture that includes cells and reagents, template particles may be provided. Template particles may be made of any suitable material such as, for example, polyacrylamide, poly (lactic-co-glycolic acid), polyethylene glycol, agarose, or other such material. In some embodiments, hydrogel particles are prepared. In some embodiments, 6.2% acrylamide (Sigma-Aldrich), 0.18% N, N′-methylene-bis-acrylamide (Sigma-Aldrich), and 0.3% ammonium persulfate (Sigma-Aldrich) are used for PAA particle generation. A total of 14% (w/v) 8-arm PEGSH (Creative PEGworks) in 100 mM NaHC03 and PEGDA (6 kDa, Creative PEGworks) in 100 mM NaHCO 3 may be used for PEG particle generation. A 1% low melting temperature agarose (Sigma-Aldrich) may be used for agarose particle generation. The agarose solution is warmed to prevent solidification. Agarose and PEG solutions are injected into a droplet generation device with the oil (FIFE-7500 fluorinated oil supplemented with 5% (w/w) deprotonated Krytox 157 FSH) using syringe pumps (New Era, NE-501). The PAA solution is injected into the droplet generation device with the fluorinated oil supplemented with 1% TEMED. The hydrogel solution and oil are loaded into separate 1 mL syringes (BD) and injected at 300 and 500 μL, respectively, into the droplet generation device using syringe pumps. The PAA and PEG droplets are collected and incubated for 1 h at room temperature for gelation. The agarose droplets are incubated on ice for gelation. After gelation, the gelled droplets are transferred to an aqueous carrier by destabilizing them in oil with the addition of an equal volume of 20% (v/v) perfluoro-1-octanol in HFE-7500. The particles are washed twice with hexane containing 2% Span-80 (Sigma-Aldrich) to remove residual oil. Following the hexane wash, the particles are washed with sterile water until all oil is removed.

In some embodiments, the template particles are provided in some form of tube or sample vessel for steps of the method 101. Any suitable vessel may be used. For example, a sample vessel may be an, e.g., 50 or 150 mL, microcentrifuge tube such as those sold under the trademark EPPENDORF. The sample vessel may be a blood collection tube such as the collection tube sold under the trademark VACUTAINER. The tube may be a conical centrifuge tube sold under the trademark FALCON by Corning Life Science. In preferred embodiments of the method, the template particles are provided in a tube within an aqueous media such as a buffer, nutrient broth, saline, or water.

A sample that contains RNA is obtained, to be added to the particles. Any suitable sample may be used. Suitable samples include environmental, clinical, library specimen, or other samples with known or unknown RNA present as cell-free RNA or present in tissue or cells (living or preserved) containing the RNA. Suitable samples may include whole or parts of blood, plasma, cerebrospinal fluid, saliva, tissue aspirate, microbial culture, uncultured microorganisms, swabs, or any other suitable sample, For example, in some embodiments, a blood sample is obtained (e.g., by phlebotomy) in a clinical setting. Whole blood may be used, or the blood may be spun down to isolate a component of interest from the blood, such as peripheral blood monocytes (PBMCs). The sample is then preferably added to a mixture such as the particles in the tube. For the method 101 it is preferable that the mixture include reagents for reverse transcription such as reverse transcriptase.

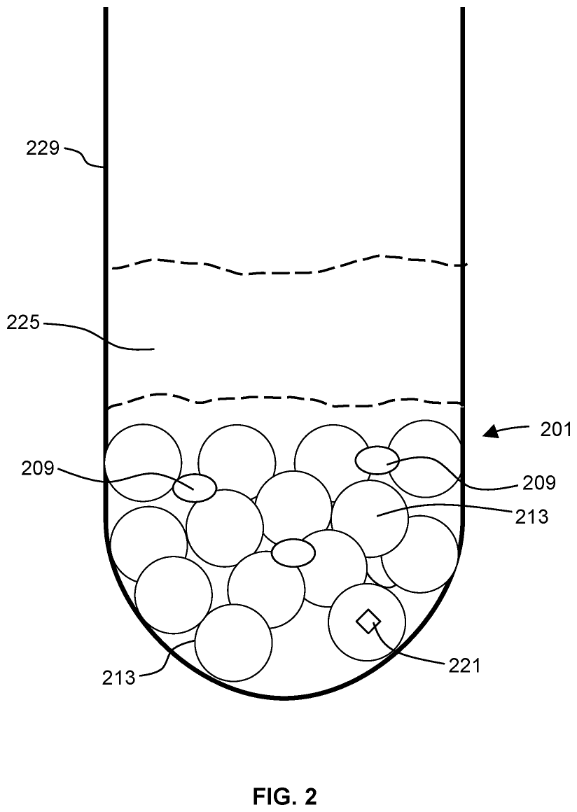

FIG. 2 shows a mixture 201 that includes cells 209 and reagents 221 for reverse transcription. As shown, the mixture 201 is provided in a sample vessel 229 or tube. The tube initially includes particles 213 that will serve as template particles for partition formation in subsequent steps. The reagents 221 may be provided by various methods or in various formats. In the depicted embodiments, the reagents 221 are provided by the particles 213. When using particles 213 of a certain structure, such as hydrogels, the reagents 221 may be enclosed within, embedded with, stuck to, or linked to the particles 213. As shown, the particles 213 and the cells 209 sit within an aqueous mixture 201. The method 101 may include adding an oil 225 onto the mixture 201 prior to any vortexing 107. It may be preferable to use a fluorinated oil for the oil 225, and a surfactant such as a fluorosurfactant may also be added (separately, or with the oil 225, or with the aqueous mixture 201). See Hatori, 2018, Particle-templated emulsification for microfluidics-free digital biology, Anal Chem 90:9813-9820, incorporated by reference. It may be found that aqueous-soluble surfactants promotes formation of monodisperse (each droplet has one particle and each particle gets a droplet) droplets. Preferred materials for the hydrogel particles 213 include polyacrylamide (PAA) and PEG. In one preferred embodiment, the sample vessel 229 includes comprise PAA particles 213 with 0.5% Triton suspended in 1.25 volume of HFE oil 225 with 2% (20 μL) or 5% (200 μL and 2 mL) fluorosurfactant. Once the aqueous mixture 201 is prepared, the mixture is vortexed.

The mixture may be vortexed by any suitable method or mechanism. The mixture may be contained in a tube such as a microcentrifuge tube. The tube may be manually flicked, or pressed down on a benchtop vortexer. The mixture may be in a well in a plate, such as a 96-well plate, and the plate may be loaded onto a benchtop mixer or shaker. The mixture may be in one tube of an 8-tube strip of microcentrifuge tubes such as the 8-tube strip sold under the trademark EPPENDORF. In a preferred embodiment, the tube is loaded into a vortexing instrument.

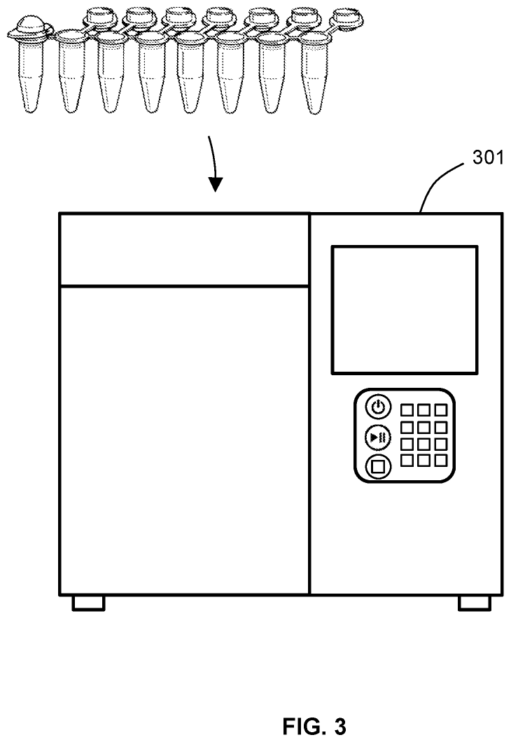

FIG. 3 shows loading an 8-tube strip into an instrument 301 for vortexing 107 the mixture (where the reaction vessel 229 is one of the 8 tubes in the strip). The instrument 301 vortexes 107 the mixture 201. During the vortexing, two things happen: droplets are generated that contain RNA and the RNA is transcribed to cDNA. The method 101 may include, during the vortexing 107, heating the mixture to a temperature that promotes activity of the reverse transcriptase. For example, the instrument 301 may include a heater that heats the sample vessel 229. The sample vessel 229 and/or reaction mixture 201 may be heated to a temperature for example between about forty and about fifty degrees C. The heating and the vortexing 107 may be performed within or on the vortexing instrument 301. Based on data shown below, preferably the vortexing instrument 301 vortexes the mixture 201 at a rate between about two hundred and about seven hundred rpm, e.g., more preferably between about 400 and 600 rpm, e.g., about 500 rpm. Within the sample vessel 229, during vortexing (or shaking, or shearing, or agitating, or mixing), each of the particles 213 preferably contain some of the reagents 221 for reverse transcription and each of the particles 213 serves as a template to initiate formation of aqueous monodisperse droplets in oil, in which each droplet comprises one particle 213.

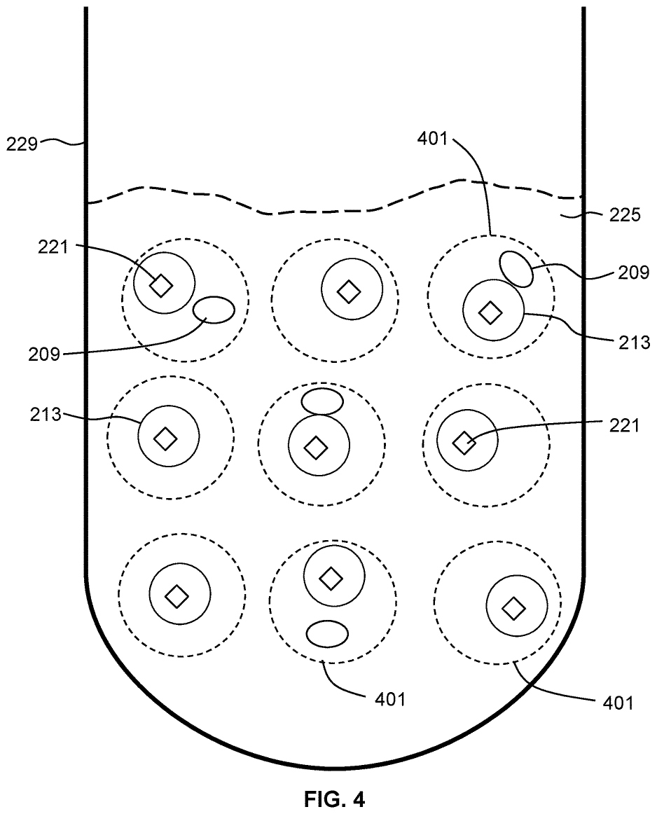

FIG. 4 shows the droplets 401 formed during vortexing 107. During the vortexing 107, the particles 213 template the formation of the droplets 401. A feature of the disclosure is that reverse transcription occurs or begins during the vortexing 107. The particles 213 and/or the mixture 201 may include reagents 221 that promote reverse transcriptions. For example, where the particles 213 are hydrogels having reagents embedded or enclosed therein, the particles may release reagents 221 into the droplets 401 as the droplets form. The particles may release the reagents as a natural consequences of forming the aqueous mixture 201 and vortexing 107 (e.g., due to osmotic or phase changes associated with introduction of an aqueous fluid, the sample, or via salts that are introduced to influence osmotic/tonic conditions. The reagents may be released by stimulus (e.g., sonication, heat, or the vortexing 107 itself). The reagents may migrate electrophoretically from the particles 213 into the surrounding aqueous media under the influence of electrostatic charge (e.g., self-repulsion out of the particles). Some or all of the reagents may be provided in or with (embedded within or surface-linked to) the particles 213 while additional or alternatively some or all of the reagents may be separately added to the sample vessel 229.

For example, in some embodiments, certain molecular reagents such as polymerase enzymes are packaged in the particles, some reagents such as oligonucleotides are linked (e.g., covalently) to the particles, and some reagents such as lysis agents (e.g., detergent), dNTPs, and metal ions are added independently.

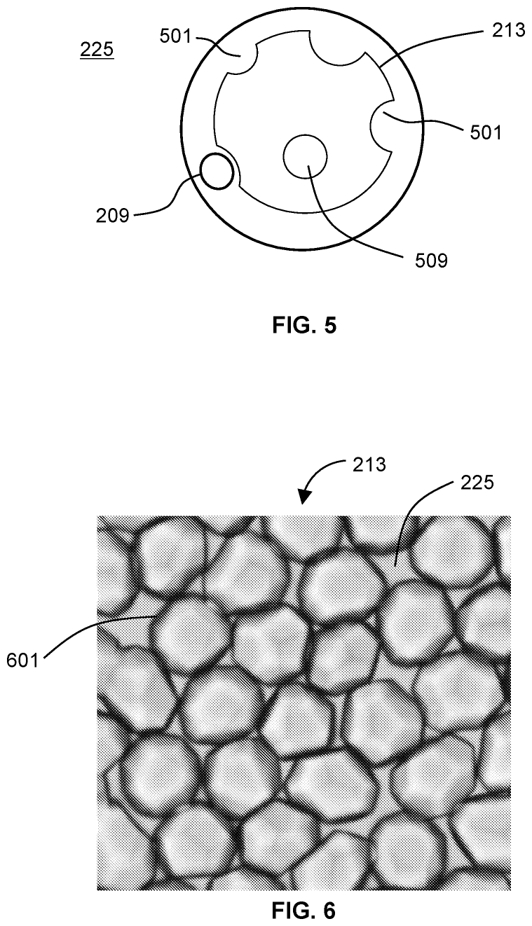

FIG. 5 is a detail view of a droplet 401 according to certain embodiments. Droplets formed according to methods of the disclosure are monodisperse meaning that the vast majority of the droplets 401 will include one particle 213 and the vast majority of the particles 213 will form into one droplet 401. Said another way, monodisperse means that comparing the number of template particles 213 initially provided in the aqueous mixture 201 to the number of droplets 401 produced by vortexing, the smaller number will be at least 90% of the larger number, and in practice usually at least 95%, more preferably 98% or 99%. Under optimal conditions, it is 99.9%. Each particle 213 may include a number of features to promote the methods herein. For example, each particle is preferably composed of a hydrogel such as poly-acryl amide (PAA). The particles may preferably be non-spherical and instead include recesses 501 or quasi-planar facets that tend to promote the association of cells 209 with the particles 213 during formation of the droplets 401 in the tube 229. Each particle 213 may include one or more of an interior void space or compartment 509 where reagents are held prior to vortexing or introduction of aqueous media. While compartments may be understood as open pockets of space having reagents therein, it may also be understood that reagents are packed into or embedded within the particles 213. It may also be found that during formation of the particles 213 that, due to electrostatic forces, water-soluble reagents migrate to a shell near an outer portion of the particle 213 and readily diffuse into aqueous media when the particle 213 is inundated therein. Other features, compositions, and morphologies are within the scope of the disclosure.

FIG. 6 is a photomicrograph showing a plurality of PAA particles 601 having quasi-planar facets. The depicted morphology may be preferred for sequestering cells into droplets. A benefit of hydrogel particles such as PAA is that methods exist for linking the particles to useful molecular structures such as oligonucleotide capture probes or primers. Covalent linkage can be provided via an acrylamide group and or through a disulfide linkage (which can be released in-droplet by providing reducing condition, e.g., by introducing beta mercaptoethanol or dithiothreitol).

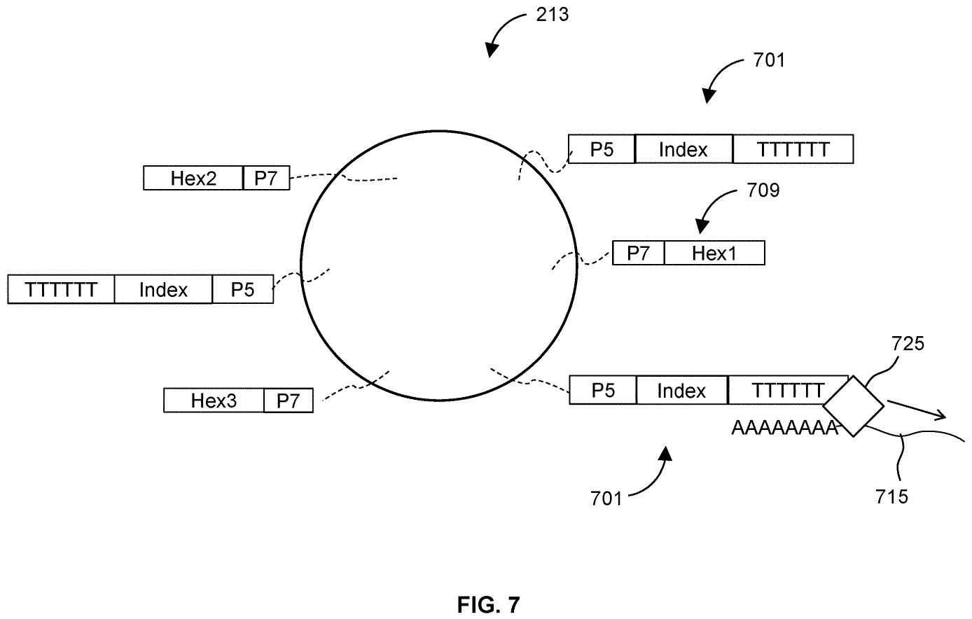

FIG. 7 shows an embodiment in which the particles 213 are linked to capture oligos usefill for initiating reverse transcription. As shown, the particle 213 is linked to (among other things) mRNA capture oligos 701 that include a 3′ poly-T region (although sequence-specific primers or random N-mers may be used). Where the initial sample includes cell-free RNA, the capture oligo hybridizes by Watson-Crick base-pairing to a target in the RNA and serves as a primer for reverse transcriptase, which makes a cDNA copy of the RNA. Where the initial sample includes intact cells, the same logic applies but the hybridizing and reverse transcription occurs once a cell releases RNA (e.g., by being lysed).

In preferred embodiments, the target RNAs are mRNAs. For example, methods of the disclosure may be used to make a cDNA library useful for showing an expression profile of a cell. Where the target RNAs are mRNAs, the particles may include mRNA capture oligos 701 useful to at least synthesize a first cDNA copy of an mRNA. The particles 213 may further include cDNA capture oligos 709 with 3′ portions that hybridize to cDNA copies of the mRNA. For the cDNA capture oligos, the 3′ portions may include gene-specific sequences or hexamers. As shown, the mRNA capture oligos 701 include, from 5′ to 3′, a binding site sequence P5, an index, and a poly-T segment. The cDNA capture oligos include, from 5′ to 3′, a binding sequence P7 and a hexamer. Any suitable sequence may be used for the P5 and P7 binding sequences. For example, either or both of those may be arbitrary universal priming sequence (universal meaning that the sequence information is not specific to the naturally occurring genomic sequence being studied, but is instead suited to being amplified using a pair of cognate universal primers, by design). The index segment may be any suitable barcode or index such as may be useful in downstream information processing. It is contemplated that the P5 sequences, the P7 sequence, and the index segment may be the sequences use in NGS indexed sequences such as performed on an NGS instrument sold under the trademark ILLUMINA, and as described in Bowman, 2013, Multiplexed Illumina sequencing libraries from picogram quantities of DNA, BMC Genomics 14:466 (esp. in FIG. 2), incorporated by reference. The hexamer segments may be random hexamers or selective hexamers (aka not-so-random hexamers). The particle 213 is depicted as including 3 hexamer segments labelled Hex 1, Hex2, and Hex3, but it will be appreciated that the particle 213 may be linked to many, e.g., thousands, of distinct hexamers. Hexamers are illustrated, but any suitable oligomers may be used.

Preferred embodiments make use of not-so-random (NSR) oligomers (NSROs). See Armour, 2009, Digital transcriptome profiling using selective hexamer priming for cDNA synthesis, Nat Meth 6(9): 647-650, incorporated by reference. Preferably, the particles 213 are linked to capture oligos 701, 709 that include one or more primer binding sequences P5, P7 cognate to PCR primers that may be used in an option downstream amplifying step (such as PCR or bridge amplification).

As shown, a capture oligo 701 hybridizes to an mRNA 715. A reverse transcriptase 725 binds and initiates synthesis of a cDNA copy of the mRNA 715. Note that the mRNA 715 is connected to the particle 213 non-covalently, by Watson-Crick base-pairing. The cDNA that is synthesized will be covalent linked to the particle 213 by virtue of the phosphodiester bonds formed by the reverse transcriptase 725.

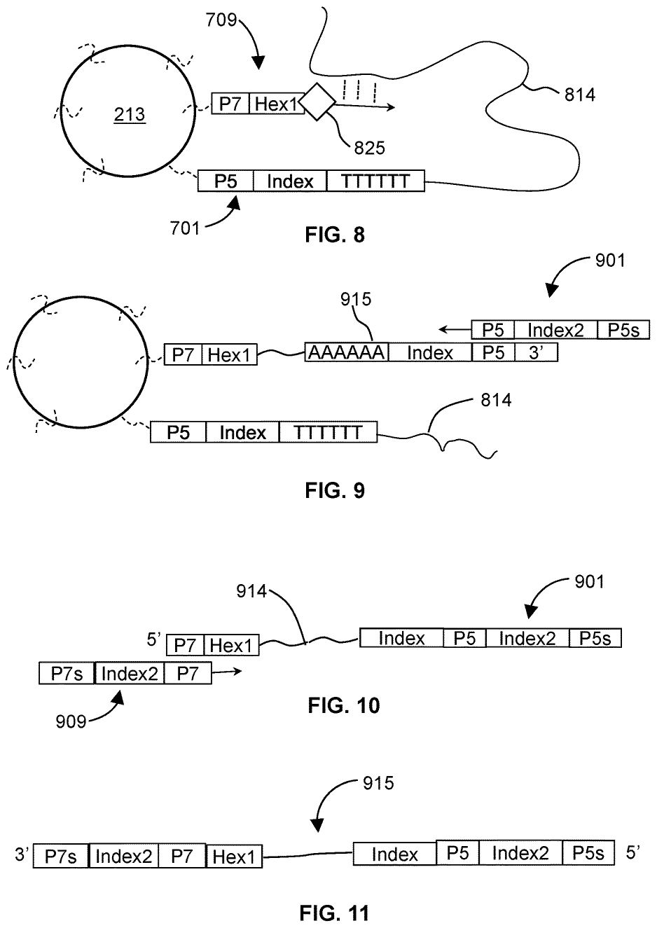

FIG. 8 shows a cDNA 814 linked to a particle by virtue of its being a covalent, polymeric extension of the mRNA capture oligo 701. As shown, a 3′ end of the cDNA capture oligo 709 will hybridize to the cDNA 814. A polymerase 825 will perform second-strand synthesis, copying the cDNA by extending the cDNA capture oligo 709.

FIG. 9 shows a first sense copy 915 of the cDNA 814. The first sense copy 915 is in the same sense as the mRNA 715, both of which are antisense to the cDNA 814. At this stage, RNaseH may be introduced to degrade the mRNA 715. A free forward primer 901 is introduced that will hybridize to, and prime copying of, the first sense copy 915 of the cDNA 814.

FIG. 10 shows the antisense copy 914 that is made by extending the free forward primer 901. A free reverse primer 909 is introduced that hybridizes to the antisense copy 914. As shown, the free forward primer 901 and the free reverse primer 909 each have respective handles P5s and P7s. Those handles P5s, P7s may be any arbitrary sequence useful in downstream analysis. For example, they may be additional universal primer binding sites or sequencing adaptors. The free reverse primer 909 primers a polymerase-based synthesis of a sense copy 915 of the original mRNA 715.

FIG. 11 shows the sense copy 915 of the original mRNA 715. It may be appreciated that the free forward primer 901, the free reverse primer 909, the antisense copy 914, and the sense copy 915 provide the basis for performing an amplification reaction. Amplifying the copies is not required and an important benefit of the disclosure is making the cDNA 814 during the vortexing 107 to form droplets 401. Because DNA is much more stable than RNA, is making the cDNA 814 during the vortexing 107 to form droplets 401 provides a convenient, useful, stable, and information-rich library for analyses such as expression analysis or sequencing.

It will be observed that copying the first sense copy 915 of the cDNA 814 using the free forward primer 901 (to produce the) is the first depicted step producing a molecular product not-covalently linked to the particle 213. Copying the sense copy 915 produces an antisense copy 914 that is not covalently linked to the particle 213. Of the sense copies 915, only the first sense copy 915 was covalently linked to the particle 213. After copying the first sense copy, every template has a barcode (“index”). This allows droplets 401 to be broken, after which multiplexing can proceed in bulk aqueous phase. In fact, where multiple droplets were formed and used to perform reverse transcription, each template strand may be barcoded by droplet. After “breaking the emulsion” (releasing contents from droplets into bulk aqueous phase), the same free forward primer 901 and free reverse primer 909 may be used to amplify, in parallel and together, any number of sense copies 915 and antisense copies 914 (each barcoded back to original droplet and optionally to individual strand).

Other variants and equivalents are within the scope of the disclosure. A feature that is preferably in common among embodiments of the disclosure is that some form of vortexing, shaking, shearing, agitating, or mixing is performed to encapsulate a plurality of particles simultaneously into droplets while some reverse transcription occurs at least partially during the vortexing, shaking, shearing, agitating, or mixing stage. Preferably, either wholly or at least in part, shaking/vortexing to form droplets is contemporaneous with synthesizing a cDNA copy of an mRNA resulting in the cDNA copy being contained within the droplet, once formed. Because methods of the disclosure are useful for making cDNAs that may serve well as samples for sequencing or quantification assays (e.g., digital PCR, for example), methods of the disclosure are useful for preparing samples where the input includes RNA.

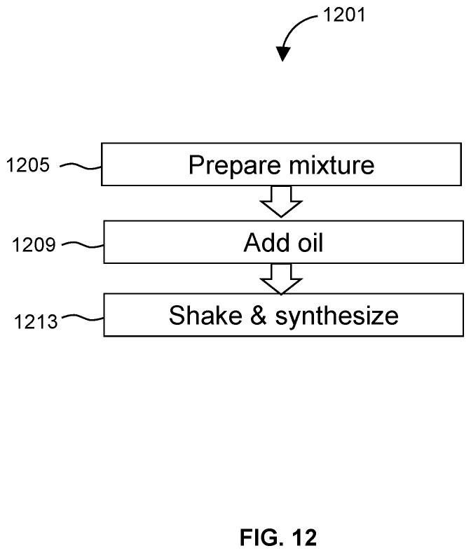

FIG. 12 diagrams a sample preparation method 1201. The method 1201 includes preparing 1205, in a sample vessel 229, an aqueous mixture 201 that includes nucleic acids (e.g., mRNA 715) and polymerase enzymes (e.g., reverse transcriptase 725). The method 1201 includes adding an oil 225 to the sample vessel 229. Further, the method 1201 includes shaking the sample vessel to partition the aqueous mixture into droplets 401 surrounded by the oil and synthesizing a DNA copy 814 of at least one of the nucleic acids with the polymerase during the shaking. The shaking and the synthesizing are performed as a single step 1213 of the method 1201. In preferred embodiments, the nucleic acids are initially in cells 209 and the shaking step forms droplets 401 that contain the cells 209 and the method includes lysing the cells 209 within the droplets 401 to release the nucleic acids (e.g., mRNA 715) into the droplets 401.

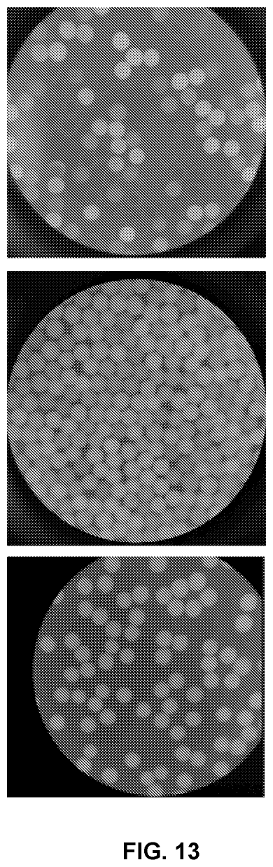

FIG. 13 shows results from performing methods of the disclosure. As shown, particles with polymerase enzymes were mixed in aqueous phase with hydrogel particles and template nucleic acids under oil and with fluorescent reagents to show polymerase activity. The top panel is a photograph of what is produced when the vessel is not subject to any mixing. The middle panel shows the results of mixing at 500 rpm. The bottom panel shows what results when mixed at 1,000 rpm. It is believed that mixing at about 500 rpm promotes the uniform formation of monodisperse droplets with simultaneous successful polymerase activity. It is believed a vortexing instrument 301 may be used to establish a uniform shearing force under about 500 rpm of motion to form monodisperse droplets. The instrument 301 may be modified to include a heater to heat the aqueous mixture 201 to an optimal temperature for the polymerase (e.g., up to about 50 degrees C). Preferably the aqueous mixture includes a plurality of template particles such as hydrogels, and shaking the sample vessel causes each template particle to serve as a template in the formation of one of the droplets. For background see WO 2019/139650 A2, incorporated by reference.

Preferably in the method 1201, the nucleic acids (e.g., mRNA 715) are initially in cells 209 and the shaking step 1213 forms droplets wherein each of the droplet 401 contains one template particle 213 and one or zero cells. The method 1201 may also include lysing the cells 209 in the droplets 401 to release the nucleic acids into the droplets. Lysing may be done by introducing a detergent such as SDS. Beneficially, the combination of shaking at about 500 rpm, the addition of SDS, and heating to about 40 to about 50 degrees C may be sufficient to lyse the cells 209. Preferably, during the shaking step, the aqueous mixture is heated to a temperature that promotes reverse transcription (e.g., about 40 to about 50 degrees C).

In some embodiments of the method 1201, the template particles are linked to capture oligos 701, linked to the template particles at their 5′ ends, wherein the 3′ ends of the capture oligos include a poly-T sequence. Each of the template particles 213 may contain some of the reverse transcriptase enzymes. During the shaking: the droplets 401 form, cells 209 are lysed within the droplets 401 to release the nucleic acids, template particles 213 capture the nucleic acids, and the polymerase enzymes synthesize the DNA copies 814. The method 1201 is suitable for the production of a plurality of monodisperse droplets where the aqueous mixture includes a plurality of template particles, and the method comprises, after the adding step, loading the sample vessel into an instrument that performs the shaking step and wherein shaking the sample vessel causes each template particle to serve as a template in the formation of one of the droplets.

The nucleic acids may initially be in cells and the shaking step forms droplets such that each of the droplets contains one template particle and one or zero cells. Preferably the nucleic acids are mRNAs in cells in the aqueous mixture, and the droplets contain the cells; and the polymerase enzymes are provided in template particles within the aqueous mixture. The method 1201 may include—after partitioning the aqueous mixture into the droplets-lysing the cells to release the mRNAs into the droplets. The template particles 2013 may be bound to capture oligos 701 that capture the mRNAs 715 and prime extension reactions by which the polymerase enzymes 725 copy the mRNAs 715.

1. A library preparation method, the method comprising:
preparing a mixture that includes cells and reagents for reverse transcription;
vortexing the mixture, wherein during the vortexing the mixture partitions into aqueous droplets that each include zero or one cell, the cells are lysed to release mRNA into the droplets, and reverse transcriptase copies the mRNA into cDNAs;
and amplifying the cDNAs into a library of amplicons.

2. The method of claim 1, wherein the mixture includes particles and wherein during vortexing the particle template the formation of the droplets, wherein the vortexing is performed using a vortexer or by pipetting to shear the mixture.

3. The method of claim 2, wherein the particles comprise gels that include the reagents therein.

4. The method of claim 2, wherein the mixture is aqueous and the method includes adding an oil onto the mixture prior to the vortexing.

5. The method of claim 2, further comprising, during the vortexing, heating the mixture to a temperature that promotes activity of the reverse transcriptase.

6. The method of claim 5, wherein the temperature is between about 40 and 50 degrees C.

7. The method of claim 2, wherein the particles are linked to capture oligos that include a 3′ poly-T region.

8. The method of claim 7, wherein the particles further include cDNA capture oligos that have 3′ portions that hybridize to cDNA copies of the mRNA, wherein the 3′ portions include gene-specific sequences or hexamers.

9. The method of claim 2, wherein the particles are linked to capture oligos that include one or more primer binding sequences cognate to PCR primers that are used in the amplifying step.

10. The method of claim 1, wherein the vortexing is performed on a vortexing instrument.

11. The method of claim 10, wherein the vortexing instrument vortexes the mixture at a rate between about 200 and 700 rpm.

12. The method of claim 10, wherein the vortexing instrument includes a heater that heats the mixture during vortexing.

13. The method of claim 2, wherein each of the particles contain some of the reagents for reverse transcription.

14. The method of claim 2, wherein each of the particles serves as a template to initiate formation of aqueous monodisperse droplets in oil, in which each droplet comprises one particle.

15. The method of claim 2, wherein during the vortexing: the mixture partitions into the aqueous droplets within about 5 to about 50 seconds, and then the cells are lysed within about 30 seconds to about a few minutes, and then the reverse transcriptase begins to copy the mRNA.

16. A sample preparation method, the method comprising:
preparing, in a sample vessel, an aqueous mixture that includes nucleic acids and polymerase enzymes; adding an oil to the sample vessel; shaking the sample vessel to partition the aqueous mixture into droplets surrounded by the oil; and
synthesizing a DNA copy of at least one of the nucleic acids with the polymerase during the shaking.

17. The method of claim 16, wherein the nucleic acids are initially in cells and the shaking step forms droplets that contain the cells, the method further comprising lysing the cells within the droplets to release the nucleic acids into the droplets.

18. The method of claim 16, wherein the nucleic acids include mRNA and the polymerase enzymes include reverse transcriptase enzymes.

19. The method of claim 16, wherein the aqueous mixture includes a plurality of template particles, wherein shaking the sample vessel causes each template particle to serve as a template in the formation of one of the droplets.

20. The method of claim 19, wherein the nucleic acids are initially in cells and the shaking step forms droplets wherein each of the droplet contains one template particle and one or zero cells, the method further comprising lysing the cells within the droplets to release the nucleic acids into the droplets.

21-30. (canceled)
